(19) United States
(12) Patent Application Publication
(10) Pub. No.: US 2026/0021071 A1
(43) Pub. Date: Jan. 22, 2026

(54) SMALL MOLECULE BROAD-SPECTRUM ANTIBIOTICS

(71) Applicant: University of Florida Research Foundation, Incorporated, Gainesville, FL (US)

(72) Inventors: Timothy Patrick SPICER, Gainesville, FL (US); Louis SCAMPAVIA, Gainesville, FL (US)

(21) Appl. No.: 18/875,755

(22) PCT Filed: Jun. 12, 2023
(86) PCT No.: PCT/US2023/025056

§ 371 Date: Dec. 17, 2024

Related U.S. Application Data
(60) Provisional application No. 63/367,367, filed on Jun. 30, 2022.

(51) Int. Cl. A61K 31/41; A61K 31/15; A61K 31/17; A61K 31/438; A61K 31/4985; A61K 31/505; A61K 31/5377

(52) U.S. Cl. CPC A61K 31/41; A61K 31/15; A61K 31/17; A61K 31/438; A61K 31/4985; A61K 31/505; A61K 31/5377

(57) ABSTRACT

Antibiotic resistance, by definition, occurs when a bacterial pathogen is no longer effectively treated with a particular drug when formerly it was. With the rise in resistance and public awareness of “superbugs,” there is a need for new antibiotics, but the number of new antibiotics in the discovery pipeline is roughly a tenth of what it was in the 1980s. Bacterial resistance has emerged, evolved, or has been transmitted to confer resistance to every marketed drug available. Multidrug resistant (MDR) infections have been identified in hospitals that are resistant to all available antibiotics. Resistance is often observed within 2 years of marketing a new antibiotic. Described herein are antibiotic compounds for use in inhibiting bacterial growth and/or treating or preventing a bacterial infection in a subject.

### CROSS-REFERENCE TO RELATED APPLICATIONS

This application claims the benefit of and priority to U.S. Provisional Patent Application No. 63/367,367, filed on Jun. 30, 2022, the entire contents of which are incorporated by reference herein.

### BACKGROUND

Antibiotic resistance, by definition, occurs when a bacterial pathogen is no longer effectively treated with a particular drug when formerly it was. With the rise in resistance and public awareness of “superbugs”, the is a need for new antibiotics, but the number of new antibiotics in the discovery pipeline is roughly a tenth of what it was in the 1980s. The primary reason is the lack of return on investment for pharmaceutical companies caused by both the typical short-term use of antibiotics and the desire of policy makers to save the best antibiotics as a last resort; ultimately keeping them on the shelf. This has led to a gap in the development of more effective and novel antibiotics. The Centers for Disease Control (CDC) monitors the spread of resistance and discloses our most serious microbial threats. As of last year, it was reported that 18 bacteria rank as a concerning, serious or urgent threat. With >2 million hospital acquired infections in America occurring yearly and ˜23,000 deaths due to resistant pathogens, the cost to our society and health care system runs in the range of tens of billions of dollars per year. The estimated cost for treatment of a single patient infected with antibiotic resistant bacteria is >$20K. Hence there is a need for novel antibiotic development.

### SUMMARY

Described herein are antibiotic compounds for use in inhibiting bacterial growth and/or treating or preventing a bacterial infection in a subject.

In some embodiments, the presently disclosed subject matter is directed to a method of inhibiting bacterial growth comprising contacting bacterial cells with a compound comprising the structure:

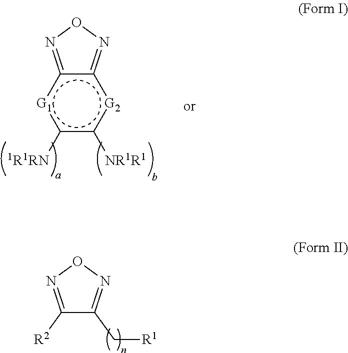

wherein:
G₁ and G₂ are N or C—R;
   wherein, R is hydrogen or an electron withdrawing group;
a and b are both independently 1 or 0;
n is an integer between 0 and 4;
each R¹ is independently H, alkyl, alkenyl, alkynyl, cyclyl, heterocyclyl, C₆-C₁₀ aryl, 5- to 10-membered heteroaryl, wherein the cyclyl, heterocyclyl, aryl or heteroaryl is optionally substituted with one, two, three, or four substituents, each of which is independently selected from the group consisting of hydrogen, C₃-C₆ cycloalkyl, phenyl, benzyl, halo-C₁-C₃ alkyl, 5- to 7-membered heteroaryl, (C₃-C₆ cycloalkyl)-alkyl, alkyl, —C(O)O-alkyl, halogen, —OR^{xa}, —CN, —C(O)NR^{xb}R^{xc}, and —NR^{xb}(CO)R^{xc};
   wherein said phenyl or 5- to 7-membered heteroaryl is optionally substituted with one or two or three substituents, each of which is independently selected from the group consisting of halogen, halo-alkyl, and alkyl;
   wherein R^{xa}, R^{xb}, and R^{xc} are selected from the group consisting of hydrogen and alkyl;
R² is a hydrogen or an electron withdrawing group;
or a pharmaceutically acceptable salt thereof.

In some embodiments, methods of inhibiting bacterial growth are described, the methods comprising contacting bacterial cells with a compound comprising the structure:

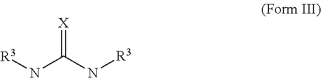

wherein:
X is independently an oxygen or a sulfur atom;
R³ is independently H, alkyl, alkenyl, alkynyl, cyclyl, heterocyclyl, C₆-C₁₀ aryl, 5- to 10-membered heteroaryl, wherein the cyclyl, heterocyclyl, aryl or heteroaryl is optionally substituted with one, two, three, or four substituents, each of which is independently selected from the group consisting of hydrogen, C₃-C₆ cycloalkyl, phenyl, benzyl, halo-C₁-C₃ alkyl, 5- to 7-membered heteroaryl, (C₃-C₆ cycloalkyl)-alkyl, alkyl, —C(O)O-alkyl, halogen, —OR^{xa}, —CN, —C(O)NR^{xb}R^{xc}, and —NR^{xb}(CO)R^{xc};
   wherein said phenyl or 5- to 7-membered heteroaryl is optionally substituted with one or two or three substituents, each of which is independently selected from the group consisting of halogen, halo-alkyl, alkyl, and —S(O)₂NHet;
   wherein Het is a 5- to 7-membered heteroaryl which is optionally substituted with one or two or three substituents, each of which is independently selected from the group consisting of halogen, halo-alkyl, and alkyl;
wherein R^{xa}, R^{xb}, and R^{xc} are selected from the group consisting of hydrogen and alkyl;
   or a pharmaceutically acceptable salt thereof.

In some embodiments, methods of inhibiting bacterial growth are described, the methods comprising contacting bacterial cells with a compound comprising the structure:

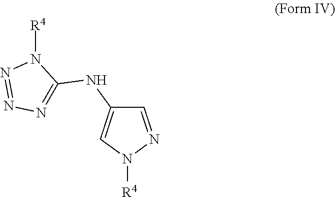

wherein:
R⁴ is independently H, alkyl, alkenyl, alkynyl, cyclyl, heterocyclyl, C₆-C₁₀ aryl, 5- to 10-membered heteroaryl, wherein the cyclyl, heterocyclyl, aryl or heteroaryl is optionally substituted with one, two, three, or four substituents, each of which is independently selected from the group consisting of hydrogen, C₃-C₆ cycloalkyl, phenyl, benzyl, halo-C₁-C₃ alkyl, 5- to 7-membered heteroaryl, (C₃-C₆ cycloalkyl)-alkyl, alkyl, —C(O)O-alkyl, halogen, —OR^{xa}, —CN, —C(O)NR^{xb}R^{xc}, and —NR^{xb}(CO)R^{xc}; wherein said phenyl or 5- to 7-membered heteroaryl is optionally substituted with one or two or three substituents, each of which is independently selected from the group consisting of halogen, halo-alkyl, alkyl, cyclyl, heterocyclyl, aryl, and heteroaryl;
   wherein R^{xa}, R^{xb}, and R^{xc} are selected from the group consisting of hydrogen and alkyl;
      or a pharmaceutically acceptable salt thereof.

In some embodiments, methods of inhibiting bacterial growth are described, the methods comprising contacting bacterial cells with a compound comprising the structure:

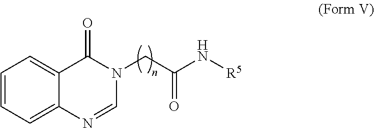

wherein:
n is an integer between 1 and 4;
R⁵ is H, alkyl, alkenyl, alkynyl, cyclyl, heterocyclyl, C₆-C₁₀ aryl, 5- to 10-membered heteroaryl, wherein the cyclyl, heterocyclyl, aryl or heteroaryl is optionally substituted with one, two, three, or four substituents, each of which is independently selected from the group consisting of hydrogen, C₃-C₆ cycloalkyl, phenyl, benzyl, halo-C₁-C₃ alkyl, 5- to 7-membered heteroaryl, (C₃-C₆ cycloalkyl)-alkyl, alkyl, —C(O)O-alkyl, halogen, —OR^{xa}, —CN, —C(O)NR^{xb}R^{xc}, and —NR^{xb}(CO)R^{xc};
   wherein said phenyl or 5- to 7-membered heteroaryl is optionally substituted with one or two or three substituents, each of which is independently selected from the group consisting of: halogen, halo-alkyl, and alkyl;
   wherein R^{xa}, R^{xb}, and R^{xc} are selected from the group consisting of hydrogen and alkyl;
      or a pharmaceutically acceptable salt thereof.

In some embodiments, methods of inhibiting bacterial growth are described, the methods comprising contacting bacterial cells with a compound comprising the structure:

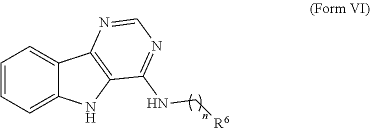

wherein:
n is an integer between 1 and 4;
R⁶ is H, alkyl, alkenyl, alkynyl, cyclyl, heterocyclyl, C₆-C₁₀ aryl, 5- to 10-membered heteroaryl, wherein the cyclyl, heterocyclyl, aryl or heteroaryl is optionally substituted with one, two, three, or four substituents, each of which is independently selected from the group consisting of hydrogen, C₃-C₆ cycloalkyl, phenyl, benzyl, halo-C₁-C₃ alkyl, 5- to 7-membered heteroaryl, (C₃-C₆ cycloalkyl)-alkyl, alkyl, —C(O)O-alkyl, halogen, —OR^{xa}, —CN, —C(O)NR^{xb}R^{xc}, and —NR^{xb}(CO)R^{xc};
   wherein said phenyl or 5- to 7-membered heteroaryl is optionally substituted with one or two or three substituents, each of which is independently selected from the group consisting of: halogen, halo-alkyl, and alkyl;
   wherein R^{xa}, R^{xb}, and R^{xc} are selected from the group consisting of hydrogen and alkyl;
      or a pharmaceutically acceptable salt thereof.

In some embodiments, methods of inhibiting bacterial growth are described, the methods comprising contacting bacterial cells with a compound comprising the structure:

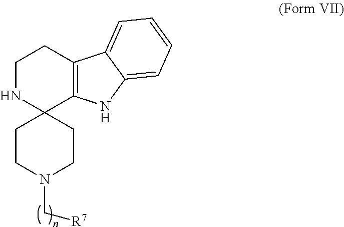

wherein:
n is an integer between 1 and 4;
R⁷ is H, alkyl, alkenyl, alkynyl, cyclyl, heterocyclyl, C₆-C₁₀ aryl, 5- to 10-membered heteroaryl, wherein the cyclyl, heterocyclyl, aryl or heteroaryl is optionally substituted with one, two, three, or four substituents, each of which is independently selected from the group consisting of hydrogen, C₃-C₆ cycloalkyl, phenyl, benzyl, halo-C₁-C₃ alkyl, 5- to 7-membered heteroaryl, (C₃-C₆ cycloalkyl)-alkyl, alkyl, —C(O)O-alkyl, halogen, —OR^{xa}, —CN, —C(O)NR^{xb}R^{xc}, and —NR^{xb}(CO)R^{xc};
   wherein said phenyl or 5- to 7-membered heteroaryl is optionally substituted with one or two or three substituents, each of which is independently selected from the group consisting of: halogen, halo-alkyl, and alkyl;
   wherein R^{xa}, R^{xb}, and R^{xc} are selected from the group consisting of hydrogen and alkyl;
      or a pharmaceutically acceptable salt thereof.

In some embodiments, methods of inhibiting bacterial growth are described, the methods comprising contacting bacterial cells with a compound comprising the structure:

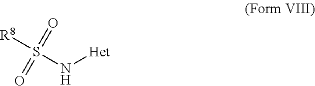

wherein:
R⁸ is H, alkyl, alkenyl, alkynyl, cyclyl, heterocyclyl, C₆-C₁₀ aryl, 5- to 10-membered heteroaryl, wherein the cyclyl, heterocyclyl, aryl or heteroaryl is optionally substituted with one, two, three, or four substituents, each of which is independently selected from the group consisting of hydrogen, C₃-C₆ cycloalkyl, phenyl, benzyl, halo-C₁-C₃ alkyl, 5- to 7-membered heteroaryl, (C₃-C₆ cycloalkyl)-alkyl, alkyl, —C(O)O-alkyl, halogen, —OR^{xa}, —CN, —C(O)NR^{xb}R^{xc}, and —NR^{xb}(CO)R^{xc};
   wherein said phenyl or 5- to 7-membered heteroaryl is optionally substituted with one or two or three substituents, each of which is independently selected from the group consisting of: halogen, halo-alkyl, and alkyl;
   wherein R^{xa}, R^{xb}, and R^{xc} are selected from the group consisting of hydrogen and alkyl;
Het is a 5- to 7-membered heteroaryl which is optionally substituted with one or two or three substituents, each of which is independently selected from the group consisting of halogen, halo-alkyl, and alkyl;
   or a pharmaceutically acceptable salt thereof.

In some embodiments, methods of inhibiting bacterial growth are described, the methods comprising contacting bacterial cells with a compound comprising the structure:

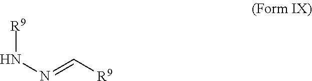

wherein:
R⁹ is H, alkyl, alkenyl, alkynyl, cyclyl, heterocyclyl, C₆-C₁₀ aryl, 5- to 10-membered heteroaryl, wherein the cyclyl, heterocyclyl, aryl or heteroaryl is optionally substituted with one, two, three, or four substituents, each of which is independently selected from the group consisting of hydrogen, C₃-C₆ cycloalkyl, phenyl, benzyl, halo-C₁-C₃ alkyl, 5- to 7-membered heteroaryl, (C₃-C₆ cycloalkyl)-alkyl, alkyl, —C(O)O-alkyl, halogen, —OR^{xa}, —CN, —C(O)NR^{xb}R^{xc}, and —NR^{xb}(CO)R^{xc};
   wherein said phenyl or 5- to 7-membered heteroaryl is optionally substituted with one or two or three substituents, each of which is independently selected from the group consisting of: halogen, halo-alkyl, and alkyl;
   wherein R^{xa}, R^{xb}, and R^{xc} are selected from the group consisting of hydrogen and alkyl;
      or a pharmaceutically acceptable salt thereof.

The bacterial cells can be in a solution, on a surface, or in a subject.

The described antibiotic compounds (e.g., the antibiotic compounds or Tables 1 and 2) of can be provided in pharmaceutical compositions or formulations. The described antibiotic compounds (e.g., the antibiotic compounds or Tables 1 and 2) or a pharmaceutical compositions or formulations thereof can be used in the prevention and/or treatment of a bacterial infection. Methods of using the described antibiotic compounds and pharmaceutical compositions and formulations thereof to treat a subject suffering from or diagnosed with, or at risk of, bacterial infection are described. In some embodiments, the described antibiotic compounds are administered to a subject to reduce one or more symptoms associated with a bacterial infection. In some embodiments, the described antibiotic compounds are administered to a subject to treat a disease associated with a bacterial infection.

The described antibiotic compounds can be formulated with one or more additional pharmaceutically active ingredients. The described antibiotic compounds can be formulated with one or more adjuvants, carriers, excipients, or a combination thereof.

In some embodiments, any one or more of the described antibiotic compounds can be administered to a subject to treat or prevent a bacterial infection, or to lessen the severity of a bacterial infection. The described compounds can be administered to a subject that has been diagnosed with a bacterial infection, is suffering from a bacterial infection, or that is at risk of acquiring a bacterial infection.

### BRIEF DESCRIPTION OF THE DRAWINGS

FIG. 1 CoA detection-based high-throughput assay. FIG. 1A Fluorescence generation upon reaction of CoA with 7-diethylamino-3-(4-maleimidylphenyl)-4-methyl coumarin (CPM). FIG. 1A Mechanism of fluorescence generation in CoA:CPM reaction.

FIG. 2 Flow diagram showing the steps involved in the FabY assay performed in 1536 well-plate format.

FIG. 3 Flow diagram showing the steps involved in the cytotoxicity assay performed in 1536-well plate format.

### DETAILED DESCRIPTION

Before describing the present teachings in detail, it is to be understood that the disclosure is not limited to specific compositions or process steps, as such may vary.

### I. Overview

The ratio of the number of essential bacteria proteins vs. targets exploited by current antibiotics is very small (3 out of ˜200; <2%). The antibiotics in use today only target a few specific pathways. This includes drugs from the following classes: beta lactams which bind penicillin-binding proteins and prevent cell wall synthesis; aminoglycosides which inhibit protein translation by inhibiting ribosomes; macrolides which also work by inhibiting protein synthesis and preventing peptide elongation; quinolones which inhibit DNA topoisomerase or gyrase; and the tetracyclines, which also inhibit protein translation at the level of 30S ribosomal subunit. Unfortunately, bacterial resistance has emerged, evolved, or has been transmitted to confer resistance to every marketed drug available. Multidrug resistant (MDR) Acinetobacter spp. infections have been identified in hospitals that are resistant to all available antibiotics. Resistance is often observed within 2 years of marketing a new antibiotic.

The current mechanisms by which bacteria elicit their resistance are diverse and numerous, but they typically arise from naturally-occurring mechanisms such as genetic mutation or transposons: i.e. acquisition from a other resistant bacteria. In addition, resistance can be very organism-specific, such as the case with Methicillin-resistant Staphylococcus aureus (MRSA), which exploits the penicillin-binding protein (PBP2a), acquired from another species: a mechanism of resistance acquisition not easily combated.

Beta-lactamases: Beta-lactamases are proteolytic enzymes produced by bacteria that irreversibly cleave the beta-lactam ring of penicillin-like cell wall synthesis inhibitors. These are commonly transmitted by multiple species. There are multiple classes of beta-lactamases. Class C are the cephalosorinases while Class A and D include the extended spectrum serine carbapenamases. There are also Class B metalloenzymes (MBL) which require divalent zinc for substrate hydrolysis. Beta-lactamase resistance to antibiotics can be overcome by modification of first-generation inhibitors, for example, cephalosporins, which are beta-lactam antibiotics targeting penicillin-binding proteins. to produce drugs that are similar yet effective again Cefoxitin is a good example, which has proven broad spectrum efficacy but also induces resistance within certain bacteria including Pseudomonas aeruginosa. This occurs through the induction of the chromosomal gene expression of beta-lactamase AmpC. In fact, there are at least five generations of marketed cephalosporin inhibitors, highlighting the effectiveness of this development strategy and also the prospects for eventual failure of this approach. Extended Spectrum Beta-Lactamase (ESBL) producing organisms confer yet another challenge as these enzymes are capable of hydrolyzing broad spectrum cephalosporins and monobactams. They are increasingly being transmitted and circulated in wide array of enteric bacteria, including E. coli. While the resistance here is well-understood, the situation is more complicated because they are typically located on transferable plasmids; i.e., they have the ability to jump from strain to strain between bacterial species. Carbapenems, including imipenem, are also beta-lactam antibiotics, but are less susceptible to the proteolytic resistance mechanisms common in bacteria, are broad spectrum and are the treatment of choice for EBSL strains. They are considered to be the last line of treatment for many Multi Drug Resistant (MDR) bacteria and often provide a window of therapeutic efficacy for most cases including MRSA and P. aeruginosa infections. Still carbapenamase resistance in Enterobacteriaceae (CREs), which is also found in Klebsiella pneumoniae. is on the rise and recognized as an urgent threat by the CDC. There is also K. pneumoniae carbepenamase resistance or KPC, a class A beta lactamase which, because of horizontal gene transfer, is not isolated to Klebsiella. In these cases, treatment options are severely limited primarily due to efficacy and or toxicity concerns often resulting in a poor clinical outcome. Finally, MBL resistant strains are also carbapenem-resistant and are most commonly translated from IMP1 or VIM2 genes. More recently, superbugs containing NDM-1 (New Delhi metallo-beta-lactamase 1) have arrived in America and are now found in circulating Enterobacteriaceae and Acinetobacter baumannii. There are no good options to treat these infections.

Membranes, Pumps and Modifying Enzymes: There are three mechanisms of bacterial resistance to aminoglycosides; 1) decreased uptake due to lowered cell permeability and/or increased efflux; 2) alterations in protein binding sites; and 3) drug modification via upregulated bacterial enzymes. Uptake of aminoglycosides is controlled by electron transport mechanisms and is particularly diminished in P. aeruginosa. Active efflux mechanisms exist in many bacteria, reducing the exposure of the antibiotic and directly limiting its effect. This is of particular interest as P. aeruginosa uses a well-known efflux pump, MexAB-OprM, while E. coli and E. cloacae use AcrAB-TolC. which can be easily exploited for drug discovery. Chemical modification is another route to resistance catalyzed by bacterial enzymes which alter the binding of the drug to its target. These include acetyltransferases which, due to evolutionary distancing from eukaryotic homologs, also represent a target for drug discovery. One such topical application inhibitor already exists, Bactroban, which selectively inactivates isoleucyl-tRNA synthetase.

Described are compounds for use as antibacterial agents. The described antibiotic compounds can be used to destroy, kill, or inhibit the growth of bacteria. In some embodiments, the compounds can be used to treat bacterial infection in a subject. In some embodiments, the described antibiotic compounds can be used to prevent bacterial growth or infection in a subject. In some embodiments, the described compositions and formulations are administered to a subject suffering from or diagnosed with a bacterial infection or at risk of developing a bacterial infection. The subject can be, but is not limited to, a mammal. The mammal can be, but is not limited to, a domestic animal or a human. In some embodiments. the described antibiotic compounds can be used to destroy, kill, or inhibit the growth of bacteria on a surface in a solution. In embodiments described herein, the compounds offer broad-spectrum anti-bacterial activity. The bacteria that can be treated with the described compounds include gram-positive and gram-negative bacteria.

In some embodiments, the described antibacterial compounds can be used as animal feed additives for promotion of growth, as preservatives in food, as bactericides in industrial application (e.g., in water-based paint and in the white water of paper mills to inhibit the growth of harmful bacteria), or as disinfectants for destroying or inhibiting the growth of harmful bacteria on a surface such as on medical or dental equipment.

In some embodiments, the compounds described herein inhibit bacterial growth via inhibition of an essential protein or signaling pathway. In some embodiments, the compounds described herein inhibit bacterial growth by inhibition of fatty acid biosynthesis

Fatty acid biosynthesis (FAB) is necessary for the production of bacterial cell walls, and therefore is essential for the survival of bacteria (Magnuson et al., 1993, Microbiol. Rev. 57:522-542). The fatty acid synthase system in E. coli is the archetypal type II fatty acid synthase system. Multiple enzymes are involved in fatty acid biosynthesis, and genes encoding the enzymes fabH, fabD, fabG. acpP, and fabF are clustered together on the E. coli chromosome. Clusters of FAB genes have also been found in Bacillus subtilis, Haemophilus influenza Rd, Vibrio harveyi, and Rhodobacter capsulatus. Examples of FAB genes in B. subtilis include fabD, yjaX, and yhfB (encoding synthase III), fabG, ywpB, yjbW, yjaY, ylpC, fabG, and acpA. The ylpC, fabG, and acpA genes are contained within a single operon that is controlled by the PylpC promoter. The FAB pathway provides the acyl groups for production of acylated homoserine lactones (HSLs). HSLs are the signaling molecules involved in quorum sensing, i.e., bacterial cell-to-cell signaling, in a wide variety of bacteria. In pathogenic bacteria, such as Pseudomonas aeruginosa, quorum sensing is a mechanism for regulating the expression of virulence factors (Hastings and Greenberg, 1999, J. Bacteriol. 181:2667-2668).

Also described are methods for identifying small molecule inhibitors of Pseudomonas aeruginosa ACP synthase III (FabY). Similar methods can be used to identify small molecule inhibitors of related (orthologous) enzymes in other bacteria.

### II. Definitions

It should be noted that, as used in this specification and the appended claims, the singular form “a,” “an,” and “the” include plural references unless the context clearly dictates otherwise. Thus, for example, reference to “a drug” includes a plurality of drugs and the like. The conjunction “or” is to be interpreted in the inclusive sense, i.e., as equivalent to “and/or,” unless the inclusive sense would be unreasonable in the context.

In general, the term “about” indicates variation in a quantity of a component of a composition not having any significant effect on the activity or stability of the composition. When the specification discloses a specific value for a parameter, the specification should be understood as alternatively disclosing the parameter at “about” that value. All ranges are to be interpreted as encompassing the endpoints in the absence of express exclusions, such as “not including the endpoints”; thus, for example, “within 10-15” or “from 10 to 15” includes the values 10 and 15. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

Unless specifically noted, embodiments in the specification that recite “comprising” various components are also contemplated as “consisting of” or “consisting essentially of” the recited components. Embodiments in the specification that recite “consisting essentially of” various components are also contemplated as “consisting of”. “Consisting essentially of” means that additional component(s). composition(s) or method step(s) that do not materially change the basic and novel characteristics of the compositions and methods described herein may be included in those compositions or methods.

As used herein, conditional language used herein, such as, among others, “can,” “could,” “might,” “may,” “e.g.,” and the like, unless specifically stated otherwise or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

An “active ingredient” is any component of a drug product intended to furnish pharmacological activity or other direct effect in the diagnosis, cure. mitigation, treatment, or prevention of disease, or to affect the structure or any function of the body of humans or other animals. Active ingredients include those components of the product that may undergo chemical change during the manufacture of the drug product and be present in the drug product in a modified form intended to furnish the specified activity or effect. A dosage form for a pharmaceutical contains the active pharmaceutical ingredient. which is the drug substance itself, and excipients, which are the ingredients of the tablet, or the liquid in which the active agent is suspended, or other material that is pharmaceutically inert. During formulation development, the excipients can be selected so that the active ingredient can reach the target site in the body at the desired rate and extent.

A “pharmacologically effective amount,” “therapeutically effective amount,” or simply “effective amount” refers to that amount (dose) of a described active pharmaceutical ingredient or pharmaceutical composition to produce the intended pharmacological, therapeutic, or preventive result. An “effective amount” can also refer to the amount of, for example an excipient, in a pharmaceutical composition that is sufficient to achieve the desired property of the composition. An effective amount can be administered in one or more administrations, applications, or dosages.

As used herein. “dose,” “unit dose,” or “dosage” can refer to physically discrete units suitable for use in a subject, each unit containing a predetermined quantity of active pharmaceutical ingredient and/or a pharmaceutical composition thereof calculated to produce the desired response or responses in association with its administration.

The terms “treat,” “treatment,” and the like, mean the methods or steps taken to provide relief from or alleviation of the number, severity, and/or frequency of one or more symptoms of a disease or condition in a subject. Treating generally refers to obtaining a desired pharmacological and/or physiological effect. The effect can be, but does not necessarily have to be, prophylactic in terms of preventing or partially preventing a disease, symptom, or condition thereof. The effect can be therapeutic in terms of a partial or complete cure of a disease, condition, symptom, or adverse effect attributed to the disease, disorder, or condition. The term treatment can include: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease and/or its symptoms or conditions. Treating can refer to therapeutic treatment alone, prophylactic treatment alone, or both therapeutic and prophylactic treatment. Those in need of treatment (subjects in need thereof) can include those already with disease or condition or those in which disease or condition is to be prevented. Treating can include inhibiting the disease, disorder, or condition, e.g., impeding its progress; and relieving the disease, disorder, or condition, e.g., causing regression of the disease, disorder, and/or condition. Treating the disease, disorder, or condition can include ameliorating at least one symptom of the particular disease, disorder, or condition, even if the underlying pathophysiology is not affected, e.g., such as treating the symptom without affecting or removing an underlying cause of the symptom.

An “analog” refers to a molecule that structurally resembles a reference molecule (e.g., an antibacterial compound) but which has been modified in a targeted and controlled manner, by replacing a specific substituent of the reference molecule with an alternate substituent. Compared to the reference molecule, an analog would be expected, by one skilled in the art, to exhibit the same or similar utility. Synthesis and screening of analogs to identify variants of known compounds having improved characteristics (such as higher binding affinity for a target molecule) is an approach that is well known in pharmaceutical chemistry.

A “derivative” of a first compound is a compound that has a three-dimensional structure that is similar to at least a part of the first compound. In some embodiments, a derivative is a compound that is derived from, or imagined to be derived from, another compound such as by substitution of one atom or group with another atom or group. In some embodiments, derivatives are compounds that at least theoretically can be formed from a common precursor compound.

As used herein, the aliphatic “alkyl”, “alkenyl” and “alkynyl” groups may be straight or branched chain having 1-10 carbon atoms; preferred are 1-6, most preferably 1-4, carbon groups; when part of another substituent, e.g., as in cycloalkylalkyl, or heteroaralkyl or aralkenyl, the alkyl, alkenyl and alkynyl group preferably contains 1-6, most preferably 1-4, carbon atoms.

As used herein, the term “heteroaryl” includes mono-, bi- and polycyclic aromatic heterocyclic groups containing 1-4 O, N or S atoms; preferred are 5- or 6-membered heterocyclic rings such as thienyl, furyl, thiadiazolyl, oxadiazolyl, triazolyl, isothiazolyl, thiazolyl, imidazolyl, isoxazolyl, tetrazolyl, oxazolyl, pyridyl, pyrazinyl, pyrimidinyl, pyridazinyl, pyrrolyl, pyrazolyl, etc.

As used herein, the term “heterocyclyl” includes mono-, bi- and polycyclic saturated or unsaturated non-aromatic heterocyclic groups containing 1-4 O, N or S atoms; preferred are 5- or 6-membered heterocyclic rings such as morpholinyl, piperazinyl, piperidyl, pyrazolinyl, pyrazolidinyl, imidazolinyl, imidazolidinyl, pyrrolinyl, pyrrolidinyl, etc.

As used herein, the term “halo” or “halogen” includes chloro, bromo, fluoro and iodo and is preferably chloro, fluoro or bromo.

As used herein, the term “electron-withdrawing group” includes nitro groups, aldehydes, ketones, cyano groups, carboxylic acids, esters, sulfate groups and halogens, and any combinations thereof, including but not limited to, trifluoromethyl groups.

The term “gram-negative bacteria” are bacteria that do not retain crystal violet-iodine complex stain due to the morphology of their cell walls. There are two major classes of gram-negative bacteria (GNB): Enterobacteriaceae and non-fermenting GNB. Examples of Enterobacteriaceae include but are not limited to Escherichia coli, Klebsiell spp., Enterobacter spp., etc. Non-fermenting bacteria include but are not limited to Pseudomonas aeruginosa, Acinetobacter baumannii, Stenotrophomonas maltophilia, etc. Gram-negative bacteria display high level resistance to most classes of antibiotics and can often be multidrug resistant.

The term “gram-positive bacteria” are bacteria that retain the crystal violet-iodine complex stain and turn purple due to lack of the outer lipid membrane present in gram-negative bacteria. Examples of gram-positive bacteria include but are not limited to Staphylococcus aureus, Streptococcus pyogenes, and Strep. pneumoniae.

### III. Antibacterial Compounds

Described are compounds with broad spectrum antibacterial activity, pharmaceutical composition containing the antibacterial compounds, and methods of using the antibacterial compounds to inhibit or prevent bacterial growth or treat a bacterial infection.

In some embodiments, methods of inhibiting bacterial growth comprise contacting a bacterial cell with a one or more of the compounds of Table 1 or Table 2. In some embodiments, methods of treating bacterial infection comprise administering to a subject an effective dose of one or more of the compounds of Table 1 or Table 2. In some embodiments, destroying, killing, preventing, or inhibiting bacterial growth on a surface or in a solution comprises treating the surface or the solution with one or more of the compounds of Table 1 or Table 2.

TABLE 1
Antibacterial Compound Classes

| Compound Class | Structure | Form |
|---|---|---|
| Oxadiazole | 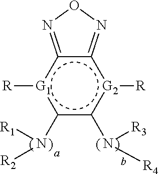 | I |
| Oxadiazole | 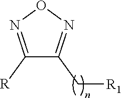 | II |
| Aryl Ureas and Thioureas | 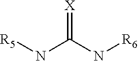 | III |
| Aminotriazoles | 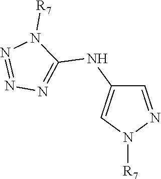 | IV |
| Pyrimidones | 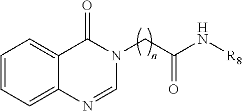 | V |
| Diazacarbazole | 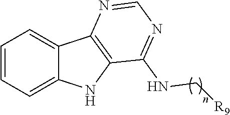 | VI |
| Spiropiperidines | 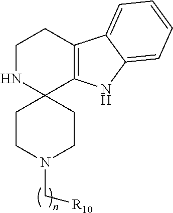 | VII |
| Biaryl sulfonamides | 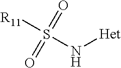 | VIII |
| FabY inhibitor | 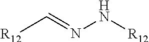 | IX |

R groups for each of the compounds in Table 1 are described above.

The compounds and methods described herein inhibit broad spectrum bacterial growth. In some embodiments, the bacterial cells are gram-positive or gram-negative bacteria. In some embodiments, the bacterial cells are gram-negative bacteria. A gram-negative bacterial can be, but is not limited to, Pseudomonas aeruginosa. In some embodiments, the bacterial cells are gram-positive bacteria. area gram-positive cell can be, but is not limited to, Staphylococcus aureus.

In embodiments, the compounds and methods described herein inhibit bacterial growth by at least about 10%, at least about 15%, at least about 25%, at least about 50%, at least about 75%, at least about 90%, at least about 95%, or at least about 99%. In some embodiments, the compounds and methods herein inhibit bacterial growth by at least about 80% or more. In some embodiments, the methods described herein result in ICso concentrations from about 0.4 μM to about 55 μM.

In some embodiments, the compounds and methods described herein can be used to inhibit bacterial growth in a subject. Inhibiting bacteria growth in a subject can be used to treat a bacterial infection. In some embodiments, the bacterial cells are in a subject. The subject ca be, but is not limited to, a domestic animal (e.g., a pet or a agriculturally important animal) or a human.

In some embodiments, the methods described herein comprise treating a subject suffering from bacterial infection or at risk of developing a bacterial infection wherein the treatment comprises administering to a subject a therapeutically effective amount of one or more compounds of Table 2.

TABLE 2
Antibacterial Compounds

| Com- | |
|---|---|
| pound | |
| No. | Structure |
| 1 | 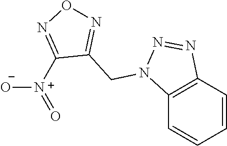 [SMILES: O=[N+]([O-])c1nonc1Cn1nnc2ccccc21] |
| 2 | 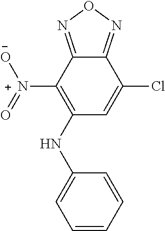 [SMILES: O=[N+]([O-])c1c(Nc2ccccc2)cc(Cl)c2nonc12] |
| 3 | 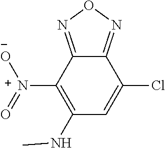 |
| 4 | 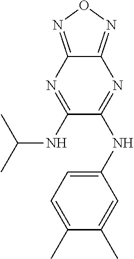 [SMILES: Cc1ccc(Nc2nc3nonc3nc2NC(C)C)cc1C] |
| 5 | 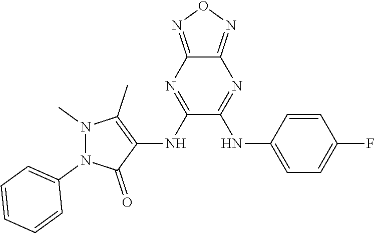 [SMILES: Cc1c(Nc2nc3nonc3nc2Nc2ccc(F)cc2)c(=O)n(-c2ccccc2)n1C] |
| 6 | 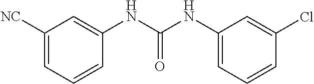 [SMILES: N#Cc1cccc(NC(=O)Nc2cccc(Cl)c2)c1] |
| 7 | 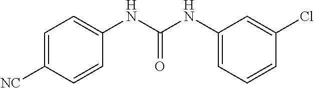 [SMILES: N#Cc1ccc(NC(=O)Nc2cccc(Cl)c2)cc1] |
| 8 | 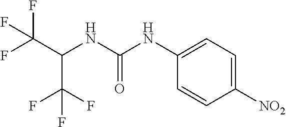 [SMILES: O=C(Nc1ccc([N+](=O)[O-])cc1)NC(C(F)(F)F)C(F)(F)F] |
| 9 | 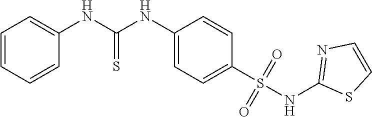 [SMILES: O=S(=O)(Nc1nccs1)c1ccc(NC(=S)Nc2ccccc2)cc1] |
| 10 | 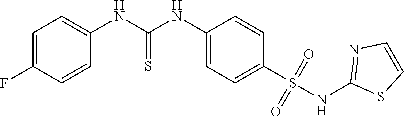 [SMILES: O=S(=O)(Nc1nccs1)c1ccc(NC(=S)Nc2ccc(F)cc2)cc1] |
| 11 | 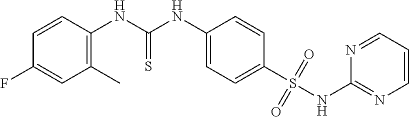 [SMILES: Cc1cc(F)ccc1NC(=S)Nc1ccc(S(=O)(=O)Nc2ncccn2)cc1] |
| 12 | 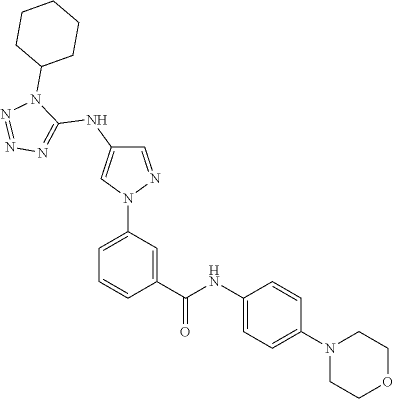 [SMILES: O=C(Nc1ccc(N2CCOCC2)cc1)c1cccc(-n2cc(Nc3nnnn3C3CCCCC3)cn2)c1] |
| 13 | 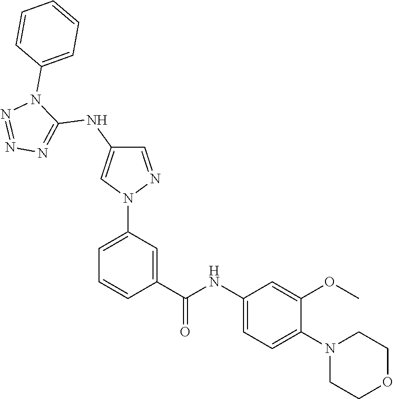 [SMILES: COc1cc(NC(=O)c2cccc(-n3cc(Nc4nnnn4-c4ccccc4)cn3)c2)ccc1N1CCOCC1] |
| 14 | 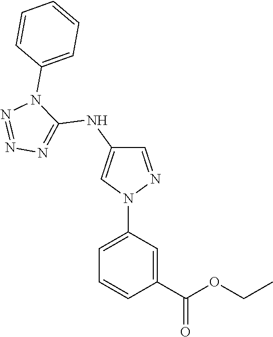 [SMILES: CCOC(=O)c1cccc(-n2cc(Nc3nnnn3-c3ccccc3)cn2)c1] |
| 15 | 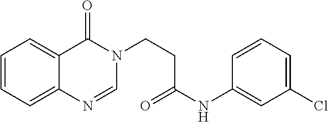 [SMILES: O=C(CCn1cnc2ccccc2c1=O)Nc1cccc(Cl)c1] |
| 16 | 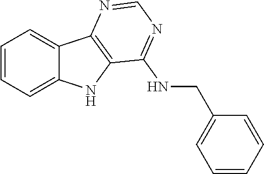 [SMILES: c1ccc(CNc2ncnc3c2[nH]c2ccccc23)cc1] |
| 17 | 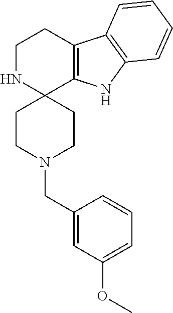 [SMILES: COc1cccc(CN2CCC3(CC2)NCCc2c3[nH]c3ccccc23)c1] |
| 18 | 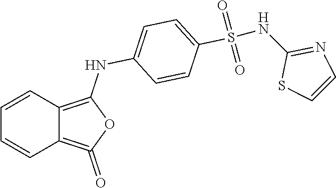 |
| 19 | 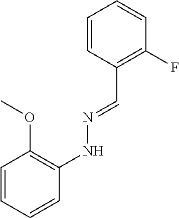 [SMILES: COc1ccccc1N/N=C/c1ccccc1F] |

### IV. Pharmaceutical Composition

In some embodiments, methods of treating a bacterial infection comprise administering to a subject a pharmaceutical composition comprising a therapeutically effective amount of one or more compounds in Table 2 or pharmaceutically acceptable salts thereof, and optionally a pharmaceutically acceptable excipient.

A “pharmaceutically acceptable excipient” refers to a vehicle for containing a functionalized cell or an acellular extracellular matrix that can be introduced into a subject without significant adverse effects and without having deleterious effects on the functionalized cell or acellular extracellular matrix. That is, “pharmaceutically acceptable” refers to any formulation which is safe and provides the appropriate delivery for the desired route of administration of an effective amount of at least one functionalized cell or acellular extracellular matrix for use in the methods disclosed herein. Pharmaceutically acceptable carriers or vehicles or excipients are well known. Descriptions of suitable pharmaceutically acceptable carriers, and factors involved in their selection, are found in a variety of readily available sources such as, for example, Remington's Pharmaceutical Sciences, 18th ed., 1990, herein incorporated by reference in its entirety for all purposes. Such carriers can be suitable for any route of administration (e.g., parenteral, enteral (e.g., oral), or topical application). Such pharmaceutical compositions can be buffered, for example, wherein the pH is maintained at a particular desired value, ranging from pH 4.0 to pH 9.0, in accordance with the stability of the functionalized cell or acellular extracellular matrix and route of administration.

Suitable pharmaceutically acceptable carriers include, for example, sterile water, salt solutions such as saline, glucose, buffered solutions such as phosphate buffered solutions or bicarbonate buffered solutions, alcohols, gum arabic, vegetable oils, benzyl alcohols, polyethylene glycols, gelatine, carbohydrates (e.g., lactose, amylose or starch), magnesium stearate, talc, silicic acid, viscous paraffin, white paraffin, glycerol, alginates, hyaluronic acid, collagen, perfume oil. fatty acid monoglycerides and diglycerides, pentaerythritol fatty acid esters, hydroxy methylcellulose, polyvinyl pyrrolidone, and the like. Pharmaceutical compositions or vaccines may also include auxiliary agents including, for example, diluents, stabilizers (e.g., sugars and amino acids), preservatives, wetting agents, emulsifiers, pH buffering agents, viscosity enhancing additives, lubricants, salts for influencing osmotic pressure, buffers, vitamins, coloring, flavoring, aromatic substances, and the like which do not deleteriously react with a functionalized cell or an acellular extracellular matrix.

For liquid formulations, for example, pharmaceutically acceptable carriers may be aqueous or non-aqueous solutions, suspensions, emulsions, or oils. Non-aqueous solvents include, for example, propylene glycol, polyethylene glycol, and injectable organic esters such as ethyl oleate. Aqueous carriers include, for example, water, alcoholic/aqueous solutions, emulsions or suspensions, including saline and buffered media. Examples of oils include those of petroleum, animal, vegetable, or synthetic origin, such as peanut oil, soybean oil, mineral oil, olive oil, sunflower oil, and fish-liver oil. Solid carriers/diluents include, for example, a gum, a starch (e.g., corn starch, pregelatinized starch), a sugar (e.g., lactose, mannitol, sucrose, or dextrose), a cellulosic material (e.g., microcrystalline cellulose), an acrylate (e.g., polymethylacrylate), calcium carbonate, magnesium oxide, talc, or mixtures thereof.

Optionally, sustained or directed release pharmaceutical compositions can be formulated. This can be accomplished, for example, through use of liposomes or compositions wherein the active compound is protected with differentially degradable coatings (e.g., by microencapsulation, multiple coatings, and so forth). Such compositions may be formulated for immediate or slow release. It is also possible to freeze-dry the compositions and use the lyophilisates obtained (e.g., for the preparation of products for injection).

The described antibiotic compounds can be formulated for oral administration, parenteral administration, IV administration, or injection. The described antibiotic compounds can be provided as a liquid formulation, as a tablet, as a coated tablet, as a chewable tablet, as a powder (e.g., a lyophilized powder), or as a capsule. Parenteral administration can be, but is not limited to, intramuscular administration and intravenous administration.

In some embodiments, any of the described antibiotic compounds can be administered in combination with or formulated in combination with one or more additional therapeutic compounds. The additional therapeutic compound can be, are not limited to, another antibiotic.

In some embodiments, the described antibiotic compounds can be administered to a subject to decrease bacteria disease burden. In some embodiments, the described antibiotic compounds can be administered to a subject to inhibit bacterial infection, decrease the likelihood of infection, decrease the severity of infection, and/or decrease the duration of infection.

Described are methods of decreasing bacterial, decreasing disease burden, preventing infection, decreasing the likelihood of infection, decreasing the severity of infection, and/or decreasing duration of bacterial infection. The methods comprise administering one or more of the described antibiotic compounds to a subject that suffering from, suspected of suffering from, or at risk of bacterial infection. In some embodiments, the methods comprise administering a pharmaceutical composition comprising an effective dose of one or more of the described antibiotic compounds to a subject that has, is suspected of having, or at risk of bacterial infection.

Described herein are compositions and formulations comprising compounds of Tables 1 and 2 for use as antibiotic compounds. The described compositions and formulations can be used in methods for therapeutic treatment and/or prevention of symptoms and diseases associated with bacterial infection. Such methods comprise administration of the compositions and formulations as described herein to a subject, e.g., a human or animal subject.

The described antibiotic compounds can be administered to a subject to prevent or treat bacteria infection in a subject. In some embodiments, the described antibiotic compounds are administered to a subject at risk of bacterial infection. In some embodiments, the described antibiotic compounds are administered to a subject that has tested positive for a bacteria injection. In some embodiments, the described antibiotic compounds are administered to a subject that has been exposed to a pathogenic bacteria. In some embodiments, the described compositions and formulations are administered to a subject at risk of being exposed to a pathogenic bacteria. In some embodiments, the described compositions and formulations are administered to a subject suffering from or diagnosed with a bacterial infection. In some embodiments, the described compositions and formulations are administered to a subject to treat one or more symptoms associated with a bacterial infection.

In some embodiments, administration of the described antibiotic compounds can be used to decrease the number, severity, and/or frequency of symptoms associated with bacterial infection in a subject.

The described pharmaceutical compositions can be used to treat at least one symptom associated with bacterial infection in a subject. In some embodiments, the subject is administered a therapeutically effective amount of one or more of the described antibiotic compounds, thereby treating the symptom. In some embodiments, the subject is administered a prophylactically effective amount of one or more of the described antibiotic compounds thereby preventing bacterial infection preventing development of one or more symptoms associated with bacterial infection.

The disclosed subject matter is further described in the following non-limiting Examples. It should be understood that these Examples, while indicating preferred embodiments of the invention, are given by way of illustration only.

### EXAMPLES

### Example 1: Whole Cell High Throughput Screening of SDDL

The whole cell phenotypic approach against the SDDL (Scripps Drug Discovery Library) (now ˜645K compounds) has already identified at least 8 diverse scaffolds of which none of the current starting points have structural overlap with existing antibiotics.

To help identify starting points for probe development initiatives, a large scale ultra-high throughput screening (uHTS) campaign was undertaken, funded in part by an externally-derived Scripps special funding proposal. As part of this effort, the PAO200 P. aeruginosa bacteria, the MexAB-OprM efflux pump minus strain of PAO1, was tested as a live/dead turbidity assay against the Scripps Drug Discovery Library (SDDL) containing >617K unique compounds. The initial intent was to focus on Gram-negatives and P. aeruginosa. This would potentially limit the number of hits found thus the efflux minus mutant of PAO1 was used for the HTS so as not to exclude possible weak inhibitors. The fully automated HTS campaign was completed in 1536-well format and was statistically robust based on Z′ as well as achieving the expected pharmacologic response to Ciprofloxacin which, had an average MIC of 0.01 μg/mL±0.01. Importantly, CLSI techniques were adhered to throughout.

After identifying an initial 1151 compounds of interest that had potency >˜7% of the sample field activity, a similar pattern of confirmation and counterscreen steps was done that used different strains of bacteria including PAO1, E. coli W3110 a TolC::Tn10 inclusive strain, and NRS135 which is the gram-positive S. aureus strain expressing NorA::tetM. Again, all assays were turbidity end point assays run using CLSI methods and all strains were monitored to achieve the anticipated Ciprofloxacin MIC on a day to day basis. All secondary and titration assays were run in triplicate and achieved excellent Z's (always >0.5).

By comparing the outcomes of this large scale HTS effort, a panel of 18 validated hits of interest were identified across 8 chemotypes; some with sub-micromolar activity (Table 3) against some of the strains and at least moderate potency against all strains tested. Generally, compounds were progressed forward at each phase of the HTS based on their ability to achieve activity above the hit cut-off criteria which was designated at >˜7% for each bacteria. Following the titration phase, best starting points were identified based on SAR and structural classes. Notably, these structural classes were compared to the known antibiotics described in the art (beta-lactams, quinolones, sulfonamides, etc.) and obvious structures that overlapped were removed from consideration. To address the issue of eukaryotic cell cytotoxicity, the top 18 compounds identified in this campaign were analyzed against all HTS campaigns they were tested in. Approximately 60 percent of the HTS campaigns tested at Scripps are mammalian cell-based assays. These assays required the cells to be alive in order for a response to be monitored. It was found that of the ˜43 cell based assays, the average number of times any analog was a hit was less than 3±2. For example, if a compound is promiscuous or highly cytotoxic then it would be anticipated to aberrantly modulate the kinetic read or end point reporter of an assay. This indicates that not only are these analogs low in their promiscuity, but they are also unlikely to be very cytotoxic to human cells.

Following the HTS effort and dose-response confirmation assays >50 possible screening hits of significant potency were identified in one or more assays. All compounds and scaffolds with tractability issues, such as having multiple undesired functional groups that are associated with toxicity, high reactivity (i.e., covalent warheads), poor drug- or lead-likeness, and pan-assay interference issues were eliminated from consideration for follow-up, after applying multiple in silico filters, including the assessment of activity in other assays previously run at the Scripps Research Institute Molecular Screening Center. Compounds and scaffolds with structural similarity to known antibiotics (for example, quinolones) were rejected. Approximately 18 hits remained for further consideration: all with reasonable potency, size, and acceptable drug-likeness. These hits fell into 8 structural classes described above in Table 1. We also biased hit selection for fragment-like properties, as demonstrated by the low average molecular weight of the eight compounds shown. All of the hit classes shown can be readily manufactured synthetically with high efficiency in 3-8 steps.

TABLE 3
Hits

| | | PAO200 | PAO01 | W3110 | NRS135 |
|---|---|---|---|---|---|
| Compound | | IC₅₀ | IC₅₀ | IC₅₀ | IC₅₀ |
| No. | Structure | (μM) | (μM) | (μM) | (μM) |
| 1 | 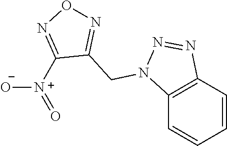 [SMILES: O=[N+]([O-])c1nonc1Cn1nnc2ccccc21] | 17.6 | 21.76 | 35.96 | 38.22 |
| 2 | 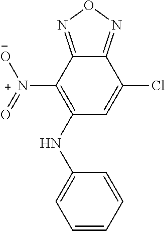 [SMILES: O=[N+]([O-])c1c(Nc2ccccc2)cc(Cl)c2nonc12] | 1.3 | 18.03 | 15.25 | 2.47 |
| 3 | 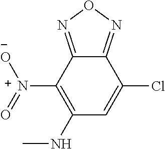 | 0.3794 | 10.17 | 12.32 | 13.04 |
| 4 | 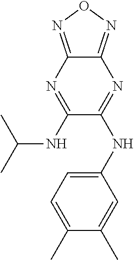 [SMILES: Cc1ccc(Nc2nc3nonc3nc2NC(C)C)cc1C] | 24.74 | 55 | 6.293 | 26.46 |
| 5 | 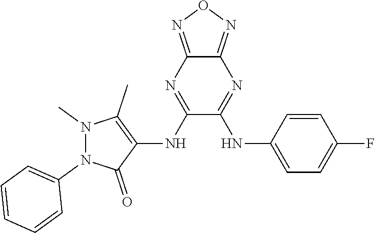 [SMILES: Cc1c(Nc2nc3nonc3nc2Nc2ccc(F)cc2)c(=O)n(-c2ccccc2)n1C] | 39.18 | 42 | 3.771 | 9.373 |
| 6 | 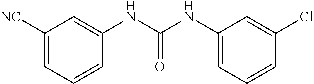 [SMILES: N#Cc1cccc(NC(=O)Nc2cccc(Cl)c2)c1] | 24.73 | 55.01 | 5.988 | 19.82 |
| 7 | 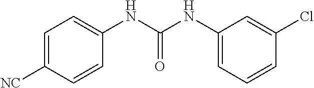 [SMILES: N#Cc1ccc(NC(=O)Nc2cccc(Cl)c2)cc1] | 24.74 | 55 | 3.82 | 9.766 |
| 8 | 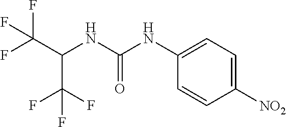 [SMILES: O=C(Nc1ccc([N+](=O)[O-])cc1)NC(C(F)(F)F)C(F)(F)F] | 31.49 | 39.53 | 8.886 | 18.11 |
| 9 | 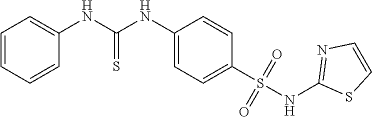 [SMILES: O=S(=O)(Nc1nccs1)c1ccc(NC(=S)Nc2ccccc2)cc1] | 12.35 | 55 | 3.798 | 21.88 |
| 10 | 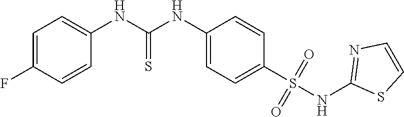 [SMILES: O=S(=O)(Nc1nccs1)c1ccc(NC(=S)Nc2ccc(F)cc2)cc1] | 12.54 | 55 | 4.002 | 17.51 |
| 11 | 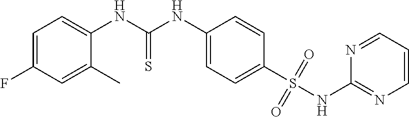 [SMILES: Cc1cc(F)ccc1NC(=S)Nc1ccc(S(=O)(=O)Nc2ncccn2)cc1] | 28.25 | 55 | 7.082 | 55 |
| 12 | 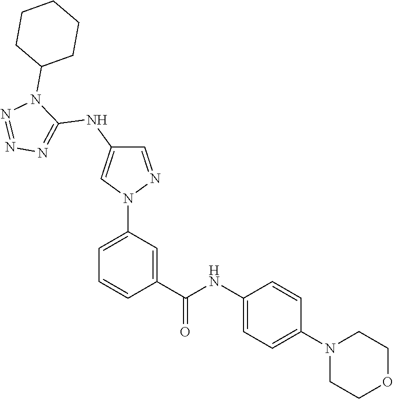 [SMILES: O=C(Nc1ccc(N2CCOCC2)cc1)c1cccc(-n2cc(Nc3nnnn3C3CCCCC3)cn2)c1] | 22.32 | 103.2 | 29.06 | 38.69 |
| 13 | 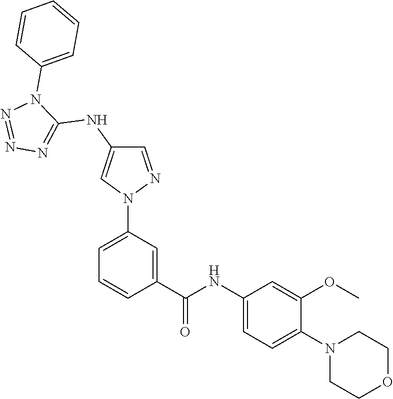 [SMILES: COc1cc(NC(=O)c2cccc(-n3cc(Nc4nnnn4-c4ccccc4)cn3)c2)ccc1N1CCOCC1] | 13.9 | 95.69 | 8.707 | 15.61 |
| 14 | 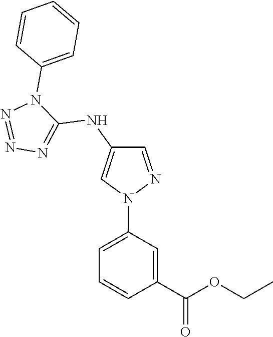 [SMILES: CCOC(=O)c1cccc(-n2cc(Nc3nnnn3-c3ccccc3)cn2)c1] | 20.46 | 55 | 14.55 | 43.63 |
| 15 | 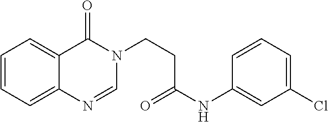 [SMILES: O=C(CCn1cnc2ccccc2c1=O)Nc1cccc(Cl)c1] | 2.701 | 38.61 | 0.8725 | 47.42 |
| 16 | 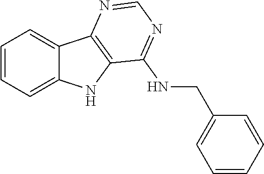 [SMILES: c1ccc(CNc2ncnc3c2[nH]c2ccccc23)cc1] | 41.49 | 55 | 15.88 | 55 |
| 17 | 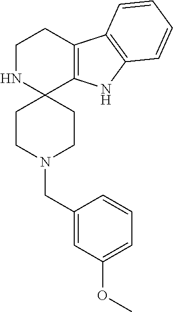 [SMILES: COc1cccc(CN2CCC3(CC2)NCCc2c3[nH]c3ccccc23)c1] | 38.95 | 28.15 | 47.42 | 55 |
| 18 | 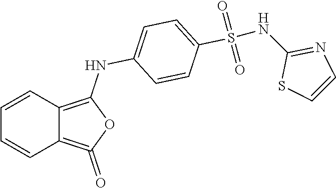 | 18.03 | 55 | 7.885 | 23.67 |
| 19 | 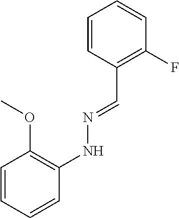 [SMILES: COc1ccccc1N/N=C/c1ccccc1F] | | | | |

As described above, hit selection was biased for lack of structural similarity to known antibiotic pharmacophores, aiming for high innovation by the discovery of novel compounds with a previously unstudied mechanism of action. All hits are commercially available, and thus SAR-by-purchase is feasible before in-house SAR efforts begin. A few of the compounds shown were initially designed for sub-libraries aimed at specific target classes, thus their activity hints at an antibacterial mechanism of action (Selwood et al., Front. Microbiol. (2018); Tiwari et al., Heterocyclic Communications 21(4):239-243 (2015)). As an example of this, the aminotetrazoles tend to be kinase inhibitors, suggesting that novel kinase in bacteria maybe a target. The spiropiperidines typically target second messenger signaling, so in this instance a quorum sensing mechanism in bacteria maybe be targeted. Both mechanisms of action would represent new opportunities in antibacterial drug design.

### Example 2: Fatty Acid Biosynthesis (FabY) Inhibition Assay

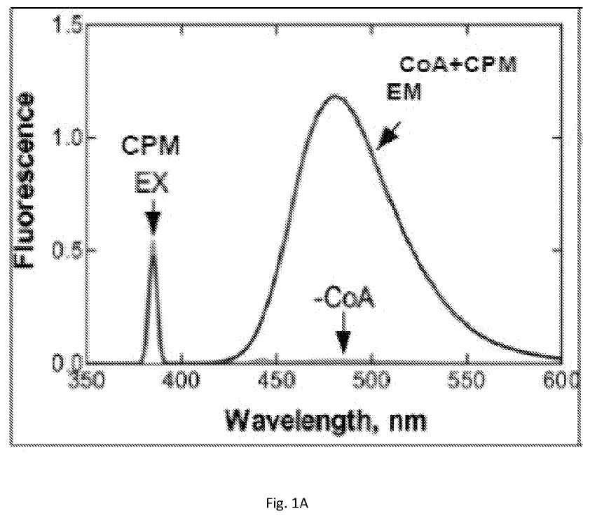

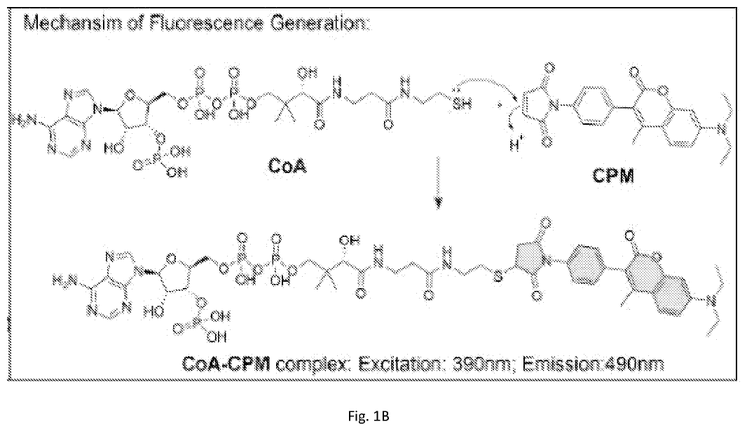

Fatty acids are assembled two carbons at a time through iterative rounds of Claisen type condensations between the malonyl-ACP donor and nascent acyl acceptor. In Pseudomonas aeruginosa. the fatty acid biosynthesis starts by the initial condensation between malonyl-ACP and acetyl co enzyme A (acetyl-CoA) to form acetoacetyl-ACP and is catalyzed by the initiating enzyme Pseudomonas aeruginosa ketoacyl ACP synthase III (FabY). Also, FabY was shown to utilize malonyl-CoA as an alternate substrate in in vitro biochemical assays. During the catalysis, FabY produces a molecule of CoA as a byproduct. To find small molecule inhibitors of this important target we have developed a new CoA detection-based high-throughput assay. The assay detects CoA, a product of the FabY-catalyzed condensation of malonyl-CoA and acetyl-CoA. The free thiol of CoA can react with 7-diethyl amino-3-(4-maleimidylphenyl)-4-methyl coumarin (CPM), a pro-fluorescent coumarin maleimide derivative that becomes fluorescent upon reaction with thiols as shown in the FIG. 1b.

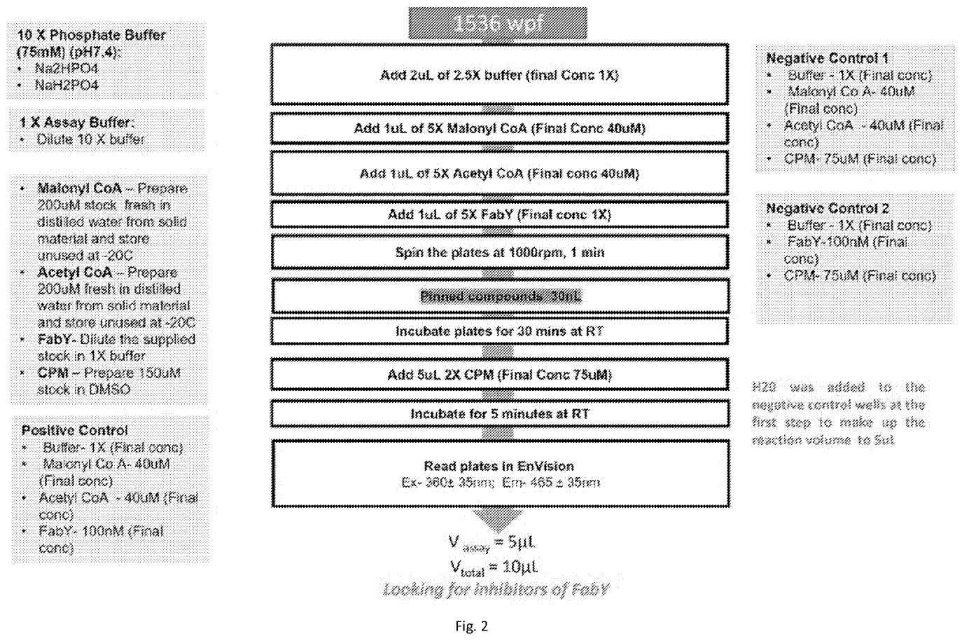

Reagent concentration/volumes were optimized and miniaturized to Greiner Bio-One 1536 well plate (Cat #789176F). The steps involved in FABY assay with the reagents, concentrations and volumes used are mentioned in the flow diagram (FIG. 2). Fluorescent intensity was read in Tecan and EnVision to ascertain reader compatibility associated with our robotics platform. The assay worked well and Z′ were passing with both negative control 1 and 2 as high controls and all reagents as low control. FIG. 1 shows the compound of interest discovered through this assay.

### Example 3: HepG2 Cytotoxicity Assay

The cytotoxicity assay uses wild-type HepG2 cells acquired from ATCC to test the cytotoxicity of the selected compounds. The assay measures cellular ATP levels as a surrogate marker of cell viability using CellTiter-Glo (Perkin Elmer). Compounds are tested at the same concentrations as used in the primary assay. Compound activity in the assay leads to a reduction in cellular ATP levels which correlates with a decrease in luminescence from the CellTiter-Glo reagent indicating cytotoxicity. Compounds are considered cytotoxic if CC₅₀ (cytotoxic concentration required to achieve 50% toxicity) is less than 10 μM. This cytotoxicity assay was used to evaluate the 16 of the 19 hit compounds described above. The cytotoxic activity of the hit compounds is described in Table 4 below.

TABLE 4
Cytotoxicity

| Cpd. | HepG2 | Avg. Max % | StdDev Max % | Hill | Ligand | Lipophilic | Lipophilic |
|---|---|---|---|---|---|---|---|
| No. | CC₅₀ (μM) | Resp. | Resp. | Slope | Efficiency | Eff. (logP) | Eff. (logD) |
| 1 | >24.8 | 36.34 | 8.65 | — | 0.34 | 2.43 | 3.71 |
| 2 | >20.0 | 5.56 | 2.31 | — | 0.33 | 0.09 | 0.09 |
| 3 | >25.4 | 9.54 | 11.80 | — | 0.43 | 2.60 | 2.60 |
| 4 | 11.4 | 70.16 | 1.11 | 11.28 | 0.31 | 1.50 | 1.50 |
| 5 | 20.6 | 80.72 | 2.18 | 11.29 | 0.20 | 1.94 | 1.94 |
| 6 | 21.1 | 94.38 | 0.43 | 14.00 | 0.34 | 1.10 | 1.10 |
| 7 | 23.8 | 94.50 | 0.50 | 18.06 | 0.34 | 1.05 | 1.05 |
| 8 | 3.2 | 81.80 | 2.37 | 12.60 | 0.35 | 2.49 | 2.49 |
| 9 | >24.8 | 1.61 | 4.93 | — | 0.26 | 0.77 | 1.41 |
| 10 | >24.8 | 5.27 | 3.14 | — | 0.25 | 0.62 | 1.27 |
| 11 | >24.8 | 3.68 | 7.81 | — | 0.23 | 0.70 | 1.29 |
| 14 | >24.8 | 4.34 | 8.60 | — | 0.23 | 1.03 | 1.03 |
| 15 | >24.8 | 4.01 | 3.78 | — | 0.28 | 1.90 | 1.90 |
| 16 | >24.8 | 30.63 | 5.50 | — | 0.31 | 1.26 | 1.26 |
| 17 | >24.8 | 8.06 | 2.83 | — | 0.24 | 1.50 | 3.51 |
| 18 | >24.8 | 3.02 | 3.88 | — | 0.25 | 1.65 | 2.13 |

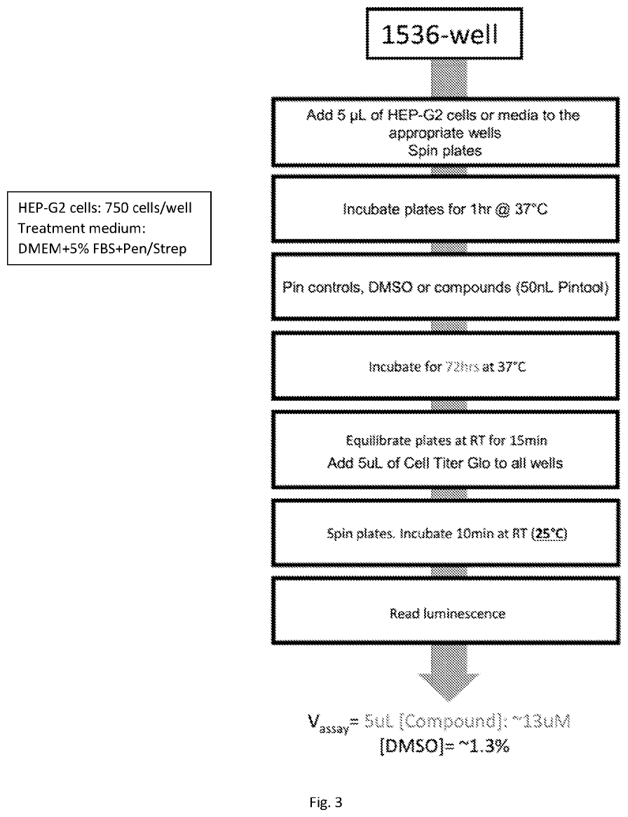

Reagent concentration/volumes were optimized and miniaturized to Greiner Bio-One 1536 well plate (Cat #789176F). The steps involved in cytotoxicity assay with the reagents, concentrations and volumes used are mentioned in the flow diagram (FIG. 3). The compounds were tested at 10 concentrations 3 times in the cytotoxicity assay. Of the 16 compounds tested, 7 compounds reached a max % response of greater than 50% and only one has an CC₅₀ of less than 10 μM (compound 8).

What is claimed is:

1. A method of inhibiting bacterial growth comprising contacting bacterial cells with a compound, wherein the compound has the structure: 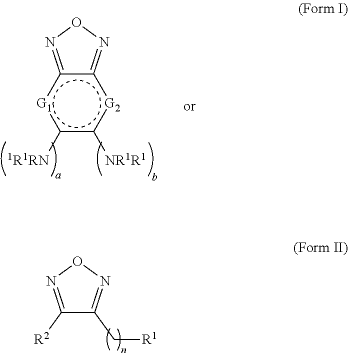
wherein:
G¹ and G² are N or C—R;
wherein, R is hydrogen or an electron withdrawing group;
a and b are both independently 1 or 0;
n is an integer between 0 and 4;
each R¹ is independently H, alkyl, alkenyl, alkynyl, cyclyl, heterocyclyl, C₆-C₁₀ aryl, 5- to 10-membered heteroaryl, wherein the cyclyl, heterocyclyl, aryl or heteroaryl is optionally substituted with one, two, three, or four substituents, each of which is independently selected from the group consisting of hydrogen, C₃-C₆ cycloalkyl, phenyl, benzyl, halo-C₁-C₃ alkyl, 5- to 7-membered heteroaryl, (C₃-C₆ cycloalkyl)-alkyl, alkyl, —C(O)O-alkyl, halogen, —OR^{xa}, —CN, —C(O)NR^{xb}R^{xc}, and —NR^{xb}(CO)R^{xc};
wherein said phenyl or 5- to 7-membered heteroaryl is optionally substituted with one or two or three substituents, each of which is independently selected from the group consisting of halogen, halo-alkyl, and alkyl;
wherein R^{xa}, R^{xb}, and R^{xc} are selected from the group consisting of hydrogen and alkyl;
R² is a hydrogen or an electron withdrawing group;
or a pharmaceutically acceptable salt thereof.

2. The method of claim 1, wherein the compound is selected from the group consisting of 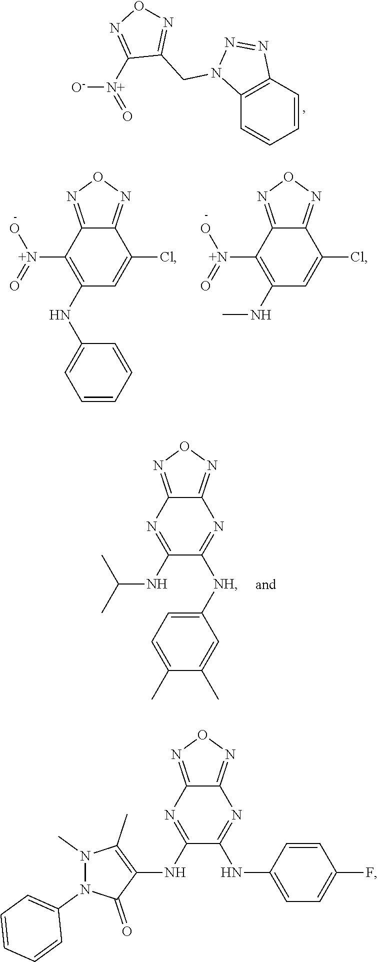
or any combination thereof.

3. A method of inhibiting bacterial growth comprising contacting bacterial cells with a compound, wherein the compound has the structure: 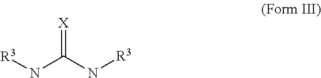
wherein:
X is independently an oxygen or a sulfur atom;
R³ is independently H, alkyl, alkenyl, alkynyl, cyclyl, heterocyclyl, C₆-C₁₀ aryl, 5- to 10-membered heteroaryl, wherein the cyclyl, heterocyclyl, aryl or heteroaryl is optionally substituted with one, two, three, or four substituents, each of which is independently selected from the group consisting of hydrogen, C₃-C₆ cycloalkyl, phenyl, benzyl, halo-C₁-C₃ alkyl, 5- to 7-membered heteroaryl, (C₃-C₆ cycloalkyl)-alkyl, alkyl, —C(O)O-alkyl, halogen, —OR^{xa}, —CN, —C(O)NR^{xb}R^{xc}, and —NR^{xb}(CO)R^{xc};
wherein said phenyl or 5- to 7-membered heteroaryl is optionally substituted with one or two or three substituents, each of which is independently selected from the group consisting of halogen, halo-alkyl, alkyl, and —S(O)₂NHet;
wherein Het is a 5- to 7-membered heteroaryl which is optionally substituted with one or two or three substituents, each of which is independently selected from the group consisting of halogen, halo-alkyl, and alkyl;
wherein R^{xa}, R^{xb}, and R^{xc} are selected from the group consisting of hydrogen and alkyl;
or a pharmaceutically acceptable salt thereof.

4. The method of claim 3, wherein the compound is selected from the group consisting of 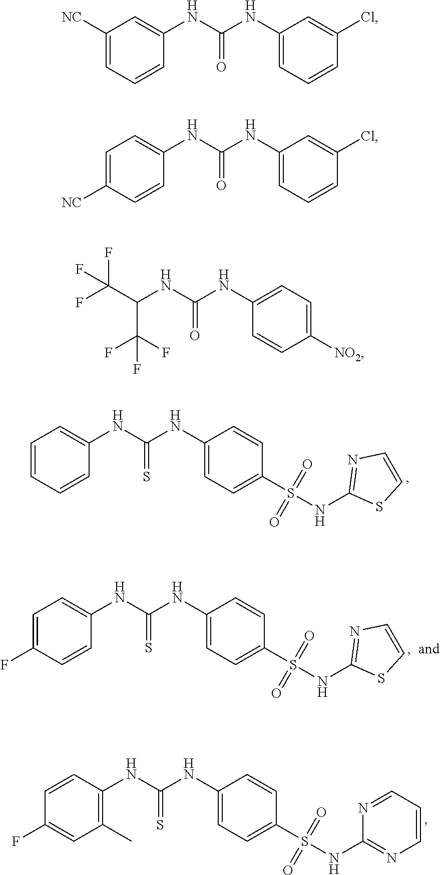 [SMILES: N#Cc1cccc(NC(=O)Nc2cccc(Cl)c2)c1 ; N#Cc1ccc(NC(=O)Nc2cccc(Cl)c2)cc1 ; O=C(Nc1ccc([N+](=O)[O-])cc1)NC(C(F)(F)F)C(F)(F)F ; O=S(=O)(Nc1nccs1)c1ccc(NC(=S)Nc2ccccc2)cc1 ; O=S(=O)(Nc1nccs1)c1ccc(NC(=S)Nc2ccc(F)cc2)cc1 ; Cc1cc(F)ccc1NC(=S)Nc1ccc(S(=O)(=O)Nc2ncccn2)cc1]
or any combinations thereof.

5. A method of inhibiting bacterial growth in a subject comprising contacting bacterial cells with a compound, wherein the compound has the structure: 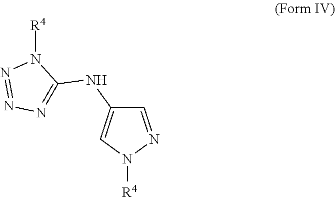
wherein:
R⁴ is independently H, alkyl, alkenyl, alkynyl, cyclyl, heterocyclyl, C₆-C₁₀ aryl, 5- to 10-membered heteroaryl, wherein the cyclyl, heterocyclyl, aryl or heteroaryl is optionally substituted with one, two, three, or four substituents, each of which is independently selected from the group consisting of hydrogen, C₃-C₆ cycloalkyl, phenyl, benzyl, halo-C₁-C₃ alkyl, 5- to 7-membered heteroaryl, (C₃-C₆ cycloalkyl)-alkyl, alkyl, —C(O)O-alkyl, halogen, —OR^{xa}, —CN, —C(O)NR^{xb}R^{xc}, and —NR^{xb}(CO) R^{xc};
wherein said phenyl or 5- to 7-membered heteroaryl is optionally substituted with one or two or three substituents, each of which is independently selected from the group consisting of halogen, halo-alkyl, alkyl, cyclyl, heterocyclyl, aryl, and heteroaryl;
wherein R^{xa}, R^{xb}, and R^{xc} are selected from the group consisting of hydrogen and alkyl;
or a pharmaceutically acceptable salt thereof.

6. The method of claim 5, wherein the compound is selected from the group consisting of 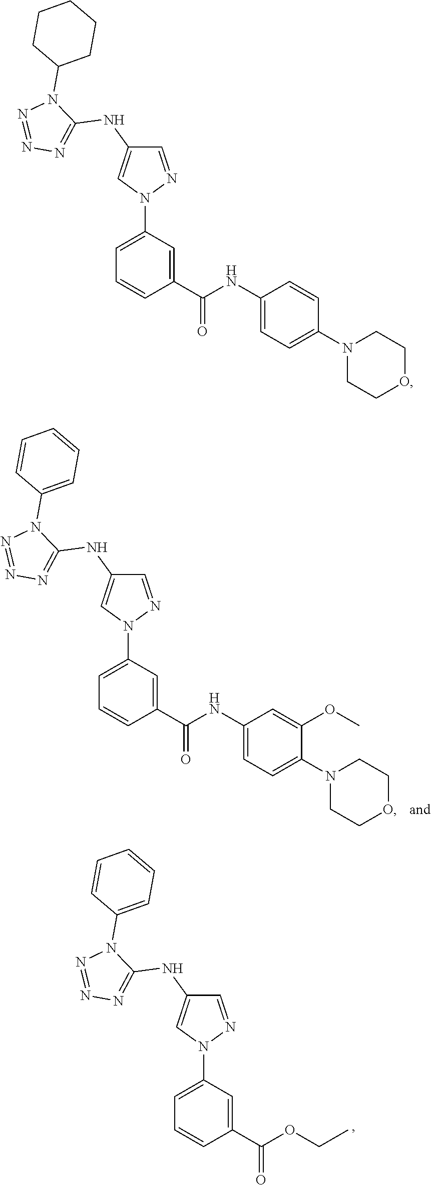 [SMILES: O=C(Nc1ccc(N2CCOCC2)cc1)c1cccc(-n2cc(Nc3nnnn3C3CCCCC3)cn2)c1 ; COc1cc(NC(=O)c2cccc(-n3cc(Nc4nnnn4-c4ccccc4)cn3)c2)ccc1N1CCOCC1 ; "and" ; CCOC(=O)c1cccc(-n2cc(Nc3nnnn3-c3ccccc3)cn2)c1]
or any combinations thereof.

7. A method of inhibiting bacterial growth in a subject comprising contacting bacterial cells with a compound, wherein the compound has the structure: 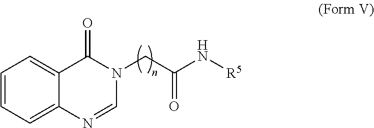
wherein:
n is an integer between 1 and 4;
R⁵ is H, alkyl, alkenyl, alkynyl, cyclyl, heterocyclyl, C₆-C₁₀ aryl, 5- to 10-membered heteroaryl, wherein the cyclyl, heterocyclyl, aryl or heteroaryl is optionally substituted with one, two, three, or four substituents, each of which is independently selected from the group consisting of hydrogen, C₃-C₆ cycloalkyl, phenyl, benzyl, halo-C₁-C₃ alkyl, 5- to 7-membered heteroaryl, (C₃-C₆ cycloalkyl)-alkyl, alkyl, —C(O)O-alkyl, halogen, —OR^{xa}, —CN, —C(O)NR^{xb}R^{xc}, and —NR^{xb}(CO)R^{xc};
wherein said phenyl or 5- to 7-membered heteroaryl is optionally substituted with one or two or three substituents, each of which is independently selected from the group consisting of: halogen, halo-alkyl, and alkyl;
wherein R^{xa}, R^{xb}, and R^{xc} are selected from the group consisting of hydrogen and alkyl;
or a pharmaceutically acceptable salt thereof.

8. The method of claim 7, wherein the compound is

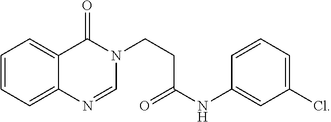 [SMILES: O=C(CCn1cnc2ccccc2c1=O)Nc1cccc(Cl)c1]

9. A method of inhibiting bacterial growth comprising contacting bacterial cells with a compound, wherein the compound has the structure: 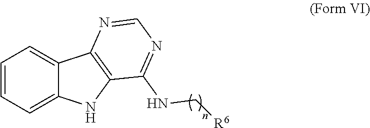
wherein:
n is an integer between 1 and 4;
R⁶ is H, alkyl, alkenyl, alkynyl, cyclyl, heterocyclyl, C₆-C₁₀ aryl, 5- to 10-membered heteroaryl, wherein the cyclyl, heterocyclyl, aryl or heteroaryl is optionally substituted with one, two, three, or four substituents, each of which is independently selected from the group consisting of hydrogen, C₃-C₆ cycloalkyl, phenyl, benzyl, halo-C₁-C₃ alkyl, 5- to 7-membered heteroaryl, (C₃-C₆ cycloalkyl)-alkyl, alkyl, —C(O)O-alkyl, halogen, —OR^{xa}, —CN, —C(O)NR^{xb}R^{xc}, and —NR^{xb}(CO)R^{xc};
wherein said phenyl or 5- to 7-membered heteroaryl is optionally substituted with one or two or three substituents, each of which is independently selected from the group consisting of: halogen, halo-alkyl, and alkyl;
wherein R^{xa}, R^{xb}, and R^{xc} are selected from the group consisting of hydrogen and alkyl;
or a pharmaceutically acceptable salt thereof.

10. The method of claim 9, wherein the compound is

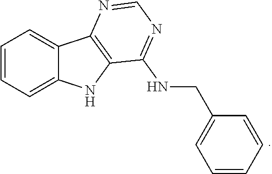 [SMILES: c1ccc(CNc2ncnc3c2[nH]c2ccccc23)cc1]

11. A method of inhibiting bacterial growth comprising contacting bacterial cells with a compound, wherein the compound has the structure: 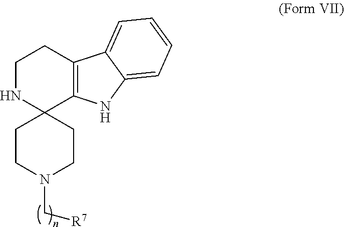
wherein:
n is an integer between 1 and 4;
R⁷ is H, alkyl, alkenyl, alkynyl, cyclyl, heterocyclyl, C₆-C₁₀ aryl, 5- to 10-membered heteroaryl, wherein the cyclyl, heterocyclyl, aryl or heteroaryl is optionally substituted with one, two, three, or four substituents, each of which is independently selected from the group consisting of hydrogen, C₃-C₆ cycloalkyl, phenyl, benzyl, halo-C₁-C₃ alkyl, 5- to 7-membered heteroaryl, (C₃-C₆ cycloalkyl)-alkyl, alkyl, —C(O)O-alkyl, halogen, —OR^{xa}, —CN, —C(O)NR^{xb}R^{xc}, and —NR^{xb}(CO)R^{xc};
wherein said phenyl or 5- to 7-membered heteroaryl is optionally substituted with one or two or three substituents, each of which is independently selected from the group consisting of: halogen, halo-alkyl, and alkyl;
wherein R^{xa}, R^{xb}, and RXc are selected from the group consisting of hydrogen and alkyl;
or a pharmaceutically acceptable salt thereof.

12. The method of claim 11, wherein the compound is

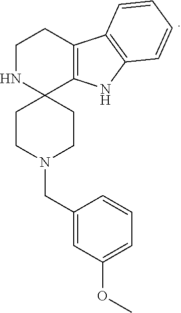 [SMILES: COc1cccc(CN2CCC3(CC2)NCCc2c3[nH]c3ccccc23)c1]

13. A method of inhibiting bacterial growth in a subject comprising contacting bacterial cells with a compound, wherein the compound has the structure: 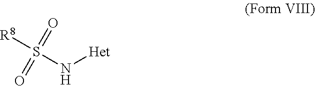
wherein:
R⁸ is H, alkyl, alkenyl, alkynyl, cyclyl, heterocyclyl, C₆-C₁₀ aryl, 5- to 10-membered heteroaryl, wherein the cyclyl, heterocyclyl, aryl or heteroaryl is optionally substituted with one, two, three, or four substituents, each of which is independently selected from the group consisting of hydrogen, C₃-C₆ cycloalkyl, phenyl, benzyl, halo-C₁-C₃ alkyl, 5- to 7-membered heteroaryl, (C₃-C₆ cycloalkyl)-alkyl, alkyl, —C(O)O-alkyl, halogen, —OR^{xa}, —CN, —C(O)NR^{xb}R^{xc}, and —NR^{xb}(CO)R^{xc};
wherein said phenyl or 5- to 7-membered heteroaryl is optionally substituted with one or two or three substituents, each of which is independently selected from the group consisting of: halogen, halo-alkyl, and alkyl;
wherein R^{xa}, R^{xb}, and R^{xc} are selected from the group consisting of hydrogen and alkyl;
Het is a 5- to 7-membered heteroaryl which is optionally substituted with one or two or three substituents, each of which is independently selected from the group consisting of halogen, halo-alkyl, and alkyl;
or a pharmaceutically acceptable salt thereof.

14. The method of claim 13, wherein the compound is

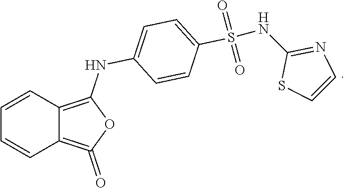

15. A method of inhibiting bacterial growth in a subject comprising contacting bacterial cells with a compound, wherein the compound has the structure: 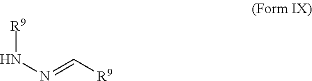
wherein:
R⁹ is H, alkyl, alkenyl, alkynyl, cyclyl, heterocyclyl, C₆-C₁₀ aryl, 5- to 10-membered heteroaryl, wherein the cyclyl, heterocyclyl, aryl or heteroaryl is optionally substituted with one, two, three, or four substituents, each of which is independently selected from the group consisting of hydrogen, C₃-C₆ cycloalkyl, phenyl, benzyl, halo-C₁-C₃ alkyl, 5- to 7-membered heteroaryl, (C₃-C₆ cycloalkyl)-alkyl, alkyl, —C(O)O-alkyl, halogen, —OR^{xa}, —CN, —C(O)NR^{xb}R^{xc}, and —NR^{xb}(CO)R^{xc};
wherein said phenyl or 5- to 7-membered heteroaryl is optionally substituted with one or two or three substituents, each of which is independently selected from the group consisting of: halogen, halo-alkyl, and alkyl;
wherein R^{xa}, R^{xb}, and R^{xc} are selected from the group consisting of hydrogen and alkyl;
or a pharmaceutically acceptable salt thereof.

16. The method of claim 15, wherein the compound is an inhibitor of the fatty acid biosynthesis pathway.

17. The method of claim 15 or claim 16, wherein the compound is an inhibitor of ketoacyl ACP synthase III (FabY).

18. The method of any one of claims 15-17, wherein the compound is

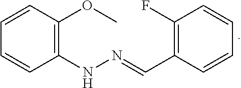 [SMILES: COc1ccccc1N/N=C/c1ccccc1F]

19. The method of any one of claims 1-18, wherein the bacterial cells are gram-positive or gram-negative bacteria.

20. The method of claim 19, wherein the bacterial cells are gram-negative bacteria.

21. The method of claim 20, wherein the bacterial cells are Psuedomonas aeruginosa.

22. The method of any one of claims 1-21, wherein the bacterial growth is inhibited about 10%, about 15%, about 25%, about 50%, about 80%, about 90%, about 95%, or about 99%.

23. The method of any one of claims 1-22, wherein the bacterial growth is inhibited about 80% or more.

24. The method of any one of claims 1-23, wherein the ICso concentration is about 0.4 μM to about 55 μM.

25. The method of any of claims 1-24, wherein the bacterial cells are in a subject.

26. The method of claim 25, wherein the subject is human.

27. The method of any one of claims 1-26, wherein inhibiting bacterial growth comprises treating a bacterial infection in a subject.

28. A method of treating a subject suffering from bacterial infection comprising administering to a subject a therapeutically effective amount of one or more compounds selected from among the group consisting of:

| Compound | |
|---|---|
| No. | Structure |
| 1 | 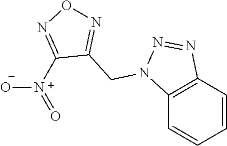 [SMILES: O=[N+]([O-])c1nonc1Cn1nnc2ccccc21] |
| 2 | 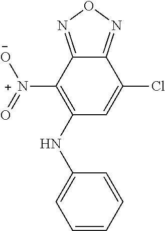 |
| 3 | 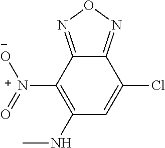 |
| 4 | 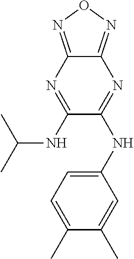 [SMILES: Cc1ccc(Nc2nc3nonc3nc2NC(C)C)cc1C] |
| 5 | 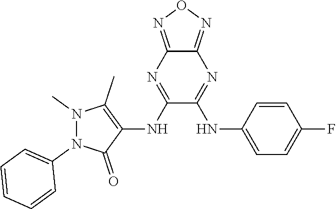 [SMILES: Cc1c(Nc2nc3nonc3nc2Nc2ccc(F)cc2)c(=O)n(-c2ccccc2)n1C] |
| 6 | 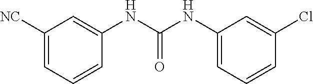 [SMILES: N#Cc1cccc(NC(=O)Nc2cccc(Cl)c2)c1] |
| 7 | 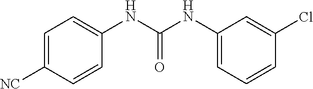 [SMILES: N#Cc1ccc(NC(=O)Nc2cccc(Cl)c2)cc1] |
| 8 | 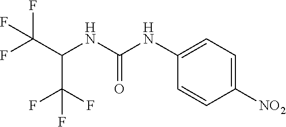 [SMILES: O=C(Nc1ccc([N+](=O)[O-])cc1)NC(C(F)(F)F)C(F)(F)F] |
| 9 | 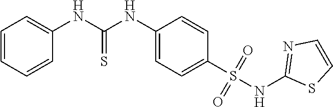 [SMILES: O=S(=O)(Nc1nccs1)c1ccc(NC(=S)Nc2ccccc2)cc1] |
| 10 | 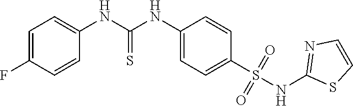 [SMILES: O=S(=O)(Nc1nccs1)c1ccc(NC(=S)Nc2ccc(F)cc2)cc1] |
| 11 | 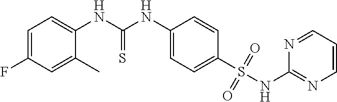 [SMILES: Cc1cc(F)ccc1NC(=S)Nc1ccc(S(=O)(=O)Nc2ncccn2)cc1] |
| 12 | 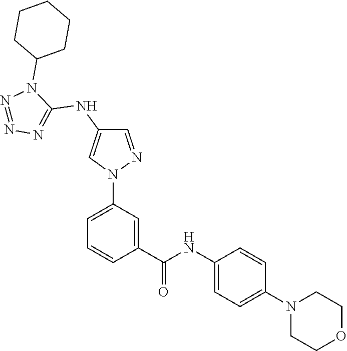 [SMILES: O=C(Nc1ccc(N2CCOCC2)cc1)c1cccc(-n2cc(Nc3nnnn3C3CCCCC3)cn2)c1] |
| 13 | 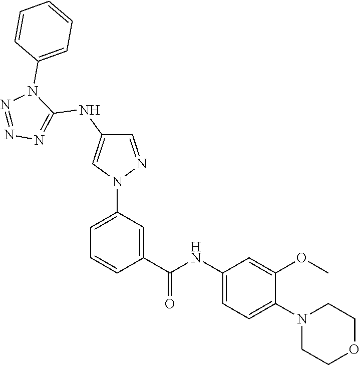 [SMILES: COc1cc(NC(=O)c2cccc(-n3cc(Nc4nnnn4-c4ccccc4)cn3)c2)ccc1N1CCOCC1] |
| 14 | 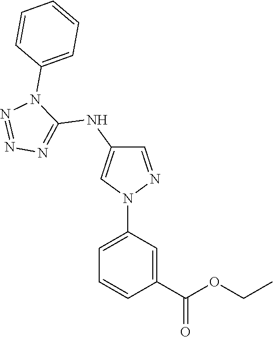 [SMILES: CCOC(=O)c1cccc(-n2cc(Nc3nnnn3-c3ccccc3)cn2)c1] |
| 15 | 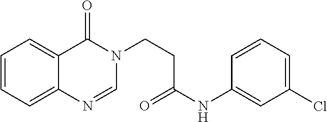 [SMILES: O=C(CCn1cnc2ccccc2c1=O)Nc1cccc(Cl)c1] |
| 16 | 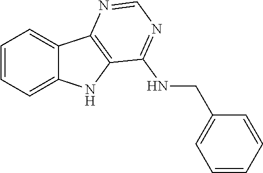 [SMILES: c1ccc(CNc2ncnc3c2[nH]c2ccccc23)cc1] |
| 17 | 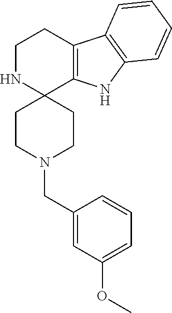 [SMILES: COc1cccc(CN2CCC3(CC2)NCCc2c3[nH]c3ccccc23)c1] |
| 18 | 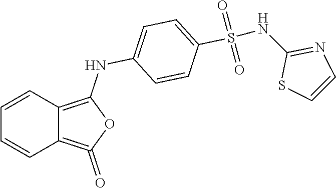 |
| 19 | 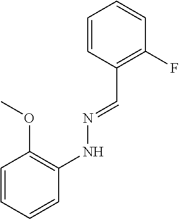 [SMILES: COc1ccccc1N/N=C/c1ccccc1F] |

29. The method of claim 28, wherein the compound is provided in a pharmaceutical composition comprising a therapeutically effective amount of the compound or pharmaceutically acceptable salt thereof, and a pharmaceutically acceptable excipient.
